(19) United States
(12) Patent Application Publication
(10) Pub. No.: US 2024/0324964 A1
(43) Pub. Date: Oct. 3, 2024

(54) Edge-intelligent Iot-based Wearable Device For Detection of Cravings in Individuals

(71) Applicants: Megan Reinhardt, Bristol, ME (US); Nicole Campagni, Reno, CA (US); Pravitha Ramanand, Cary, NC (US)

(72) Inventors: Megan Reinhardt, Bristol, ME (US); Nicole Campagni, Reno, CA (US); Pravitha Ramanand, Cary, NC (US)

(21) Appl. No.: 18/738,755

(22) Filed: Jun. 10, 2024

Related U.S. Application Data
(63) Continuation-in-part of application No. 17/857,342, filed on Jul. 5, 2022.
Application No. 17/857,342 is a continuation-in-part of application No. 16/688,861, filed on Nov. 19, 2019, now Pat. No. 11,375,896.

(51) Int. Cl. A61B 5/00; A61B 5/16; G16H 40/67; G16H 50/70

(52) U.S. Cl. CPC A61B 5/7267; A61B 5/0022; A61B 5/165; A61B 5/681; A61B 5/7282; A61B 5/746; G16H 40/67; G16H 50/70

(57) ABSTRACT

An edge-intelligent Internet based wearable device improves stress and craving detection by monitoring and interpreting a subject's feedback to alerts in the real world. The subject receives alerts physiological signals in the real world after leaving a treatment facility. Alerts are provided to the subject, and the subject indicates if the feel stress or cravings. The subject's indication is

### CROSS-REFERENCE TO RELATED APPLICATIONS

The present application is a Continuation In Part of U.S. patent application Ser. No. 17/857,342 filed Jul. 5, 2022, which is a Continuation In Part of U.S. patent application Ser. No. 16/688,861 filed Nov. 19, 2019, which is a Continuation In Part of U.S. patent application Ser. No. 15/681,111 filed Aug. 18, 2017, which applications are incorporated in their entirety herein by reference.

### BACKGROUND OF THE INVENTION

The present invention relates to detection of destructive stress or cravings in subjects, and in particular a method for improved personalized detection of stress or cravings in subjects using the subjects feedback to refine the detection of stress or cravings after forming initial classification rules.

Drug addiction is an increasingly serious individual and societal issue. The rate of drug addiction in the United States has reached levels where this addiction affects not only the addict, but society as a whole. Many addicts recognize their personal damage due to the use of drugs and desire to overcome their addiction through various programs. The subjects often successfully complete a program, but are not able to remain drug free after re-entering society, especially when they are subject to the stress that both existed in the past, and stress produced by social issues they encounter when they attempt to rejoin society. Such stress often results in cravings to resume drug use. Other similar conditions include eating disorders/food addiction, co-occurring disorders with addiction, and Post-Traumatic Stress Disorder (PTSD). A need exists for identifying the presence of stress and the resulting cravings to allow intervention before a return to drug use.

Using known technologies, the cloud which that has large servers with enormous computing, control and storage capacity, serves as the backbone for all the internet based wearable devices. Because of the increase in the number of connected wearable devices, the data traffic to and from the Internet of Things (IoT) cloud increases data redundancy and delay in processing. Further, known commercially available wearable sensors only monitor an subject's heart rate and activity and may not provide sufficient measurements to support detection of stress likely to cause cravings and facilitate refinement of stress or cravings after leaving a treatment center.

### BRIEF SUMMARY OF THE INVENTION

The present invention addresses the above and other needs by providing a method for improving accuracy of the detection of stress or cravings in subjects, after determining initial classification rules, using data clustering methods. The clustering methods may be applied before leaving the treatment center and/or later in the real world after leaving the treatment facility. With the growth of interrelated systems of computing devices, mechanical and digital machines, objects, animals or people connected by the Internet, there is a significant interest in the use of wearable sensors such as smart watches and smart phones. These wearable sensors may be used to monitor physiological signals and provide health information. The wearable senors provides the alerts to the subject and allows the subject to confirm or deny the presence of stress or cravings, The subject's response is then used to improve the classification rules.

In accordance with another aspect of the invention, there is provided a system develop initial classification rules to detect an individual subject's stress or cravings, and use of drugs, based on physiological sensors, advanced signal processing and a machine learning framework, at a treatment facility. A wearable sensor suite is worn by a subject recovering from drug use. The sensor suite produces signals by measuring physiological parameters such as one, two, or three dimensions of body movement (locomotor activity), Electro Dermal Response (EDR), heart rate, skin temperature, a galvanic skin response. Statistical data (e.g., the mean and variance) of the measurements are computed. The statistical features may be used to assess stress and/or cravings in the individual subject. Data has shown that accelerometer measurements in each dimension are significantly but not perfectly (less than 100%) correlated with other dimensions, and hence the detection of stress or cravings can be achieved with one dimension data with reasonable accuracy without EDR or temperature data. The physiological data is processed in windows having a length L, for example, a five minute window.

In accordance with still another aspect of the invention, there is provided a system to determine shape and scale parameters of a distribution of amplitudes of the three dimensional movement data are computed. Amplitudes, frequencies, and phases of the three dimensions of motion signals may be obtained using an appropriate transform. The distribution of amplitudes provides a sensitive measure capable of detecting the frequency of use of drugs (heavy use vs. moderate use). Dynamic features such as instantaneous fluctuations of amplitudes, frequencies, and phases at multiple time scales may be obtained by the time-frequency decomposition of these signals using an appropriate transform, for example the Hilbert or a wavelet transform approach.

In accordance with yet another aspect of the invention, there is provided a method for adapting a drug use risk detection method to an individual subject to develop initial classification rules at a treatment facility. A training data set comprising statistical and dynamic features is collected and incorporated in a machine learning framework. The data is collected over a one to two day period where the subject is monitored to ensure that there is no drug use. The processed three dimensional motion signals, the EDR, and temperature signals, are processed by machine learning algorithms to establish boundaries for non-drug use. The machine learning framework is tailored specifically to individual subjects to assess pathological fluctuations in the physiological signals that can be used later assess the risk or return to drug use.

In accordance with still another aspect of the invention, there is provided a method for applying clustering to improve the initial classification rules.

In accordance with another aspect of the invention, there is provided a method for detection of stress or cravings. Following release of the individual subject from a treatment facility, the individual subject is provided with a wearable device measuring physiological data. The wearable device continuously measures physiological signals, and the signals are processed for relevant features related to stress or cravings. An alert is provided to the individual subject and/or a provider through of any imminent risk of using illicit drugs, stress or cravings. By accurately tracking the statistical and dynamic fluctuations in these physiological signals in real time, the method can provide accurate detection of stress or cravings.

In accordance with yet another aspect of the invention, there is provided a method for providing alerts to a subject or a care giver. The present system includes non-invasive wearable biosensors that may stream data continuously in real time to a processor which processes the physiological signals and executes a craving or risk detection software. Once a specific threshold of risk has reached, the algorithm can trigger an alert through a smart phone to the subject or the care giver.

In accordance with still another aspect of the invention, there is provided a method for providing alerts based on a 16 dimension vector space of ten physiological signals comprising mean and variance of three dimensional motion, EDR, and temperature, and six spatial features comprising shape and scale of histogram data.

In accordance with another aspect of the invention, there is provided a wearable sensor monitoring three dimensional data at a 32 samples per second, EDR data at four samples per second, and temperature data at one sample per second. The data is provided in windows of about 5 minute length.

In accordance with still another aspect of the invention, there is provided a monitoring system including a wearable sensor, a smart phone type device, and a monitoring facility. A subject's data collected by the wearable sensor may be processed in the wearable sensor, in the smart phone type device, or at the monitoring facility. In some embodiments, the wearable sensor and smart phone type device may be a single device.

In accordance with another aspect of the invention, there is provided a wearable sensor. The wearable sensor includes physiological sensors and is an edge-intelligent device processing sensor measurements and sending assessments of stress or cravings to a remote sever accessed by clinicians.

In accordance with yet another aspect of the invention, there is provided a method for a wearable sensor sensing and processing subject data during supervision at a treatment facility. The method includes collecting training data, processing the training data, applying machine learning to the training data and subject inputs to determine classification rules, and transmitting the training rules to a cloud/clinicians.

In accordance with still another aspect of the invention, there is provided a method for a wearable sensor sensing and processing subject data after release from supervision at the treatment facility. The method includes repeating the method used during supervision, but also building a deep neural network in the cloud or at clinician facilities, and providing alerts to the subject's smart phone.

In accordance with another aspect of the invention, there is provided an edge intelligent unit which can process data at the wearable device thereby avoiding delay for critical time-sensitive applications. There are several design and implementation features in the edge intelligent unit provided to perform the required computations to the wearable. These include: operating the proposed framework using wireless communication instead of connected internet like the cloud services; using a battery-operated wearable device to perform the computation; integrating machine learning algorithms in real-time which requires training and testing phase; and providing feedback to the subject or healthcare providers without using a mobile application and smart phone.

In accordance with another aspect of the invention, there is provided a method for personalizing classification rules for detection of cravings and stress in subjects using the wearable sensor system in the real world after leaving the treatment facility. The method accounts for subject variability inherent in perception of mental states such as stress or cravings. Known methods use a machine learning framework at the treatment facility to develop classification rules for physiological data collected by the device and send alerts to the subject when stress or craving are detected. After leaving the treatment facility (in the field), the subject responds to the alerts by confirming or denying the presence of stress or cravings. This information is utilized in the present method to improve the classification rules for stress or cravings based on the subject's inputs in the field. The present invention thus improves the classification rules in the field.

In accordance with yet another aspect of the invention, there is provided a method addressing challenges encountered in real world application of stress and craving detection. Some of the challenges are: 1. individual subject's variability in physiological response to stress and craving in a real world environment, 2; and changes to an individual subject's response to stress and cravings over time. The initial classification rules are based on responses from a time restricted period at the treatment facility, and subjects are likely to respond differently to the same stimuli/events when out in the field leading daily lives. A personalized methodology based on experiencing stress and craving events in the field differentiate subject responses and captures subject specific behavior in real world environments, leading to improved classification rules for each subject. This provides better event detection for multi demographic diverse subjects related to substance abuse or other scenarios when personalized systems are used.

In accordance with still another aspect of the invention, there is provided a clustering method comparing alerts based on the initial classification rules applied to physiological data, to the subject's perception of the events, to form clusters of event detections. When the subject confirms or denies an alerted event based on the initial classification rules, thereby agreeing or disagreeing with presence of the event detected by the initial classification rules, the clustering method develops clusters from the subject's recognized events. The sensor data, for example accelerometer, heart rate and inter-beat interval signals, is characterized by a set of features estimated in the statistical, spectral, and non-linear domains to comprehensively capture the individual subject's physiological response. A clustering technique, such as k-means method, is applied to these features and the subject-responded events characterized by this technique. The clustering comprises mapping the events to a graph with the estimated features as the axis, like identifying a place on earth on a two-dimensional map of the earth with latitude and longitude values. However, unlike the two dimensions for locating places, in this case, the dimension of the space in which events are located will be based on the number of features determined which are three or more. As more responses of an individual subject to the detected events are compiled, these will identify the regions in the feature space where the confirmed events are localized. This feature space region may vary between subjects based on their physiological response levels to stress or craving. By focusing only on the regions of interest in the feature space for each subject, personalized detection of stress or craving events is carried out. This methodology leads to better accuracy of event detection and the features can be used to train personalized machine learning models at this stage for individual subjects for detection of stress or craving events.

In accordance with another aspect of the invention, there is provided a clustering, or machine learning methodology of data mining, which groups data based on their similarities and differences. Only the raw data is available as input to the model and no labels/classes/categories into which data best fits into, are provided during the training of the model. K-means clustering is one of the clustering methods used frequently in data analysis in which input data are assigned to K-groups or clusters based on its features. K represents the number of clusters that each data point is assigned to based on its distance from the center point in the feature space, also known as the centroid of the group. Data points that are closest to a centroid will belong to the same cluster whereas data points that are farther away will belong to another cluster. The training method includes arriving at the optimal assignment of the data points to the K-groups based on the structures and patterns contained in the features characterizing the data. K-means clustering is commonly used in market segmentation, document clustering, image segmentation, and image compression.

An example of clustering often mentioned in the literature is that of customer segmentation which a store wants to carry out for a targeted marketing campaign. The store has data on customer age, income, shopping frequency, spending score, number of items bought per visit and other relevant information. These constitute the features characterizing the customer data. Using these features, a clustering methodology will be to initially split customers into, for example, K=3 groups and to identify which group constitutes the most potential for responding to a marketing initiative. For this, the features are represented mathematically either as numerical (age, income) or categorical (e.g. shopping frequency: once a week, once a month etc.) and centroids of the K clusters are randomly assigned. Then an iterative method in which the distance between the data points in the same cluster in the feature space, and the cluster centroid, is minimized while the distance between data points in different clusters is maximized. This process is carried out mathematically, to arrive at an optimal distribution of the data points in the feature space to the predefined number of K clusters, in this case K=3. As the model is developed and analyzed, the data may suggest that with 3 clusters, there is too much grouping and loss of granularity. Hence, K=5 may be the optimal number of clusters suitable for the data, with each group having a set of features that are common/similar for customers within that group but different from those in another cluster. Group 1 may comprise young shoppers who spend a lot but shop occasionally, while group 2 may be older customers who spend moderately but shop more frequently. This is the training/development and evaluation phase of the clustering model. As new customers get added to the database (test data), the store can assign them to the previously developed customer clusters and efficiently target the chosen group for marketing campaigns. In the marketing example, there are 5 features for the data that result in clusters in 5-dimensional feature space.

In accordance with still another aspect of the invention, there is provided a personalized event detection algorithm where each data segment corresponds to accelerometry, heart rate and heart rate variability signals characterized by a set of features descriptive of various aspects of the sensor signals. These features are used to cluster the segments in an N-dimensional feature space, such that segments with similar features will be closer or part of the same cluster. The clusters are identified which confirmed stress or cravings of an subject are assigned to. These cluster assignments may vary based on the subject physiological response and experience of the craving/stress event. Hence by detecting only those events that fall into determined craving or stress related clusters, personalization is achieved.

In accordance with another aspect of the invention, there is provided a clustering technique for identifying feature space regions of personalized events initiates when a sizable number of events and their subject responses have been acquired (for example, 100 events). The sensor data features are computed for each detected event with a response, and these are used to cluster all the detected events for a subject into a pre-specified number of clusters. The features corresponding to data related to an event are assigned to the cluster with which it is most similar based on a ‘distance’ metric chosen appropriately for the applied clustering method. The response data is combined with this, to identify the clusters into which the subject's confirmed stress or cravings are assigned to and these regions will be specific for the subject.

The method optimizes subjective classification rules based on the clustering behavior of physiological signal features associated with confirmed events. The localization of the confirmed events starts as soon as a few events are detected and it is continuously updated as more events are detected and responded to by the subject, making this method a real time process. When the classification of events into clusters has been optimized based on the evaluation of separability criteria of clusters of different event groups, detected events will generate alerts only if they occur in the pre-specified clusters of the feature space. Further, the confirmed events for the subject will be leveraged to develop a personalized machine learning model for craving detection which may be hosted either on the wearable or in the cloud.

In accordance with yet another aspect of the invention, there is provided a method including a novel set of wearable sensor data features used for identifying the clusters in the personalized algorithm. An initial set comprising of features such as shape and scale parameter characterizing the instantaneous amplitude of signals was used to develop the event detection machine learning model as described in previous patents. These are estimated in addition to the initial set because to understand the inter subject variabilities, other aspects of the sensor data need to be characterized and compared.

### BRIEF DESCRIPTION OF THE SEVERAL VIEWS OF THE DRAWING

The above and other aspects, features and advantages of the present invention will be more apparent from the following more particular description thereof, presented in conjunction with the following drawings wherein:

FIG. 1 shows components of a system for sensing physiological parameters and detecting stress or cravings based on the physiological parameters in subjects experiencing stress or cravings, according to the present invention.

FIG. 2 shows a method for processing the subject physiological parameters to detected stress or cravings during supervised learning, according to the present invention.

FIG. 3 shows a method for processing the subject physiological parameters measured during an unsupervised period after the supervised training to detect stress, cravings, or drug use, according to the present invention.

FIG. 4 shows a plot of a training spectrum, a monitoring spectrum, and a threshold, according to the present invention.

FIG. 5 is a wearable sensor, according to the present invention.

FIG. 6 shows components of a wearable system for sensing physiological parameters and detecting stress or cravings based on the physiological parameters in subjects with addiction, according to the present invention.

FIG. 7 shows components of a microcontroller of the wearable system, according to the present invention.

FIG. 8 shows data acquisition and processing during the supervised training phase at the microcontroller, according to the present invention.

FIG. 9 shows a method for collecting and processing a subject's physiological parameters during the supervised training phase, according to the present invention.

FIG. 10 shows a method for collecting and processing a subject's physiological parameters after the supervised training phase and providing alerts, according to the present invention.

FIG. 11A show a first method for personalizing detection of stress or cravings in subjects after completing the supervised training using the wearable sensor system, according to the present invention.

FIG. 11B shows a second method for personalizing detection of stress or cravings in subjects after completing the supervised training using wearable sensor system, according to the present invention.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

Where the terms “about” or “generally” are associated with an element of the invention, it is intended to describe a feature's appearance to the human eye or human perception, and not a precise measurement.

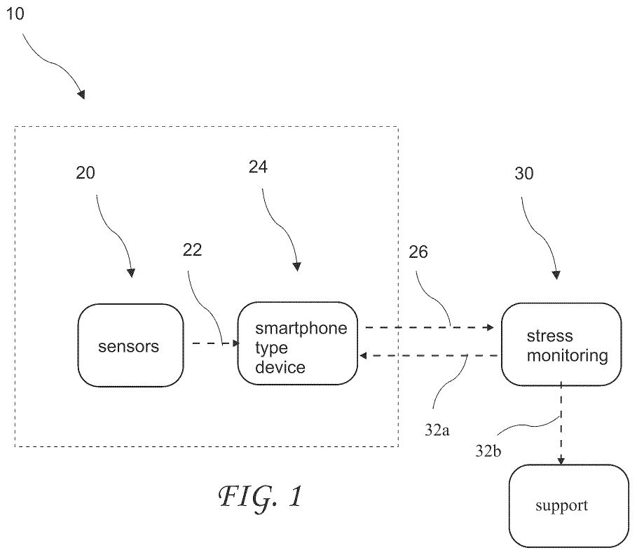

Components of a system according to the present invention for detection of stress or cravings in subjects with addiction are shown in FIG. 1. The system includes wearable devices 10 comprising sensor suite 20 and a smart phone type device 24 carried by the subject. The sensor suite 20 measures physiological parameters including three dimensional body movement, Electro Dermal Response (EDR), and temperature. A suitable sensor suite and/or method is described in U.S. patent application Ser. No. 17/857,342 filed Jul. 5, 2022, U.S. patent application Ser. No. 16/688,861 filed Nov. 19, 2019, and U.S. patent application Ser. No. 15/681,111 filed Aug. 18, 2017 The sensor suite 20 includes at least one accelerometer, a temperature sensor, and EDR sensor. The '342, 861, and 111 applications are incorporated by reference in their entirety into the present specification.

The sensor suite 20 preferably wirelessly communicates with a smart phone type device 24 to provide data 22 to the smart phone type device 24. The wireless communication may be, for example, Bluetooth communication. While Bluetooth is a preferred wireless interface, those skilled in the art will recognize other types of communication, including wired, and a system according to the present including any form of communication between the sensors and the smart phone type device, and the sensor suite 20 and smart phone type device 24 may be a single device.

The smart phone type device 24 receives the three dimensional movement, the EDR, and temperature signals 22 from the sensor suite 20 and transits the data 26 to a stress monitoring center 30. If the stress or cravings exceed a threshold, or advanced processing indicates a craving, the stress monitoring center 30 may provide alerts 32a back to the subject and alerts 32b to support personnel. The data may be processed in the sensor suite 20, the smart phone type device 24, or at the stress monitoring center 30, and the processing may be distributed over the sensor suite 20, the smart phone type device 24, and the stress monitoring center 30. Those skilled in the art will recognize that any distribution of the method between devices is intended to come within the scope of the present invention.

A method for processing the subject physiological parameters measured during a period of supervised no drug use at a treatment facility to establish initial classification rules is shown in FIG. 2. The method includes wearing a sensor device to collect a training data set for supervised learning at step 100, sensing three dimensional motion, Electro Dermal Response (EDR), and temperature training data over a one to two day period of supervised no drug use by the subject at step 102, determining the mean and variance of the training data at step 104, transforming the three dimensional motion training data into amplitude data at step 106, creating a histogram of the transformed data at step 108, fitting a curve to the histogram data at step 110, determining shape and scale from the curve fit at step 112, applying machine learning to the mean variance, shape and scale data to establish classification rules at step 114.

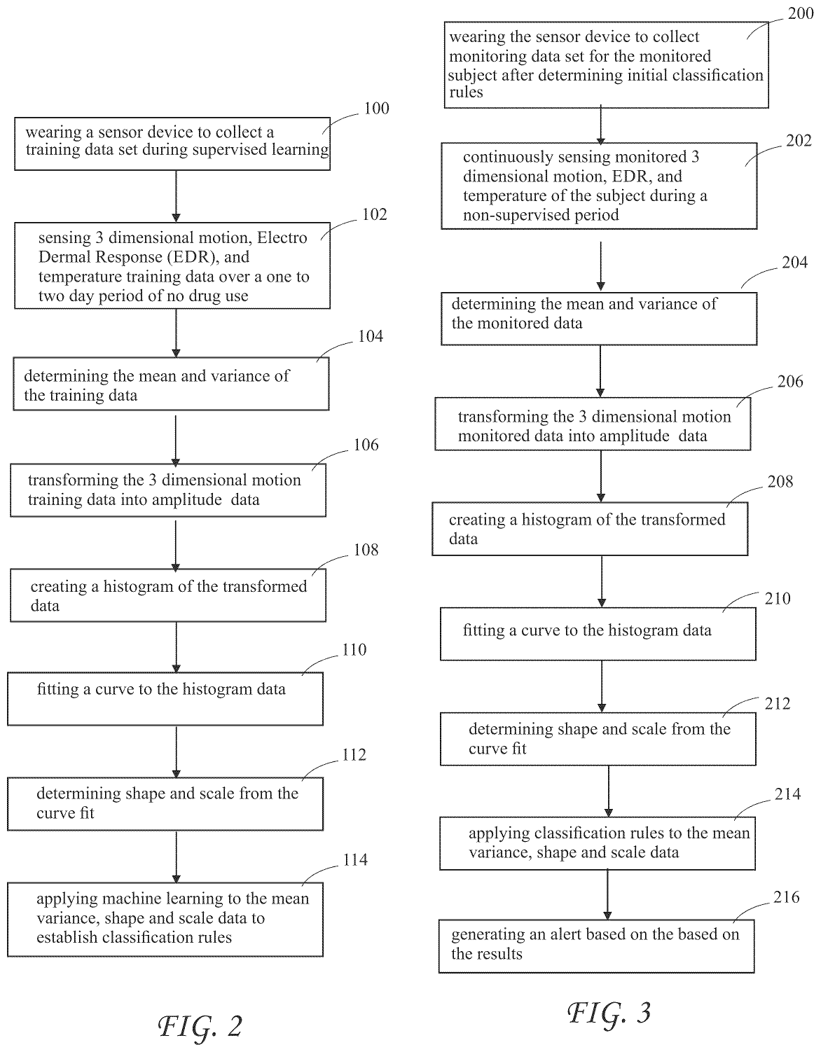

A method for processing the subject's physiological parameters measured during an unsupervised period following the supervised training to detect stress, cravings, and/or drug use, is shown in FIG. 3. The method includes wearing the sensor device to collect monitoring data set for the monitored subject after determining initial classification rules at step 200, continuously sensing monitored three dimensional motion, EDR, and temperature of the subject during a non-supervised period is shown in step 202, determining the mean and variance of the monitored data is shown in step 204, transforming the three dimensional motion monitored data into amplitude data is shown in step 206, creating a histogram of the transformed data is shown in step 208, fitting a curve to the histogram data is shown in step 210, determining shape and scale from the curve fit is shown in step 212, applying classification rules to the mean variance, shape and scale data is shown in step 214, and generating an alert based on the results is shown in step 216. The alerts may be provided to the subject and/or to a monitor and may be an alert for stress, cravings, and/or drug use. Examples of transforms used in steps 106 and 206 are a Hilbert transform or a wavelet transform. Examples of curves applied to the curve fit of steps 110 and 210 are fitting a gamma function to the amplitude data.

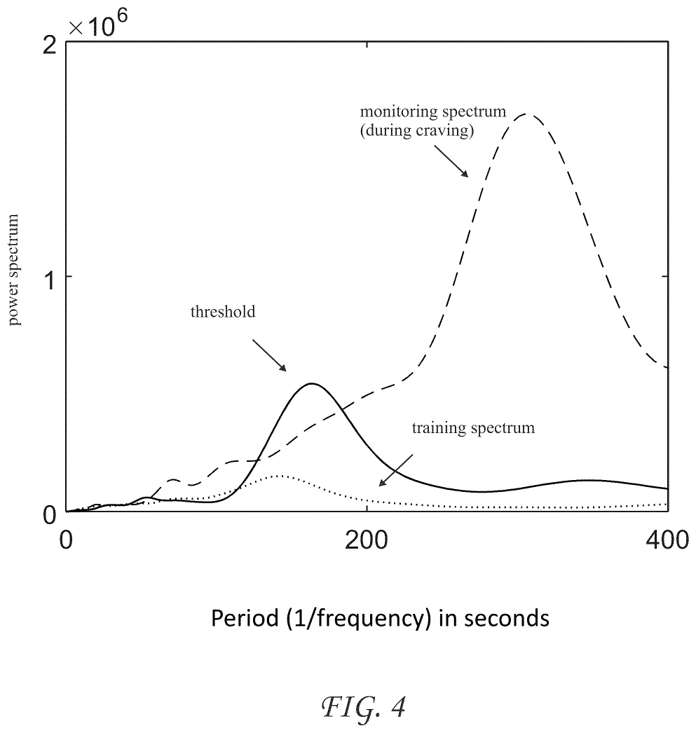

A plot of a training spectrum, a monitoring spectrum, and a threshold are shown in FIG. 4.

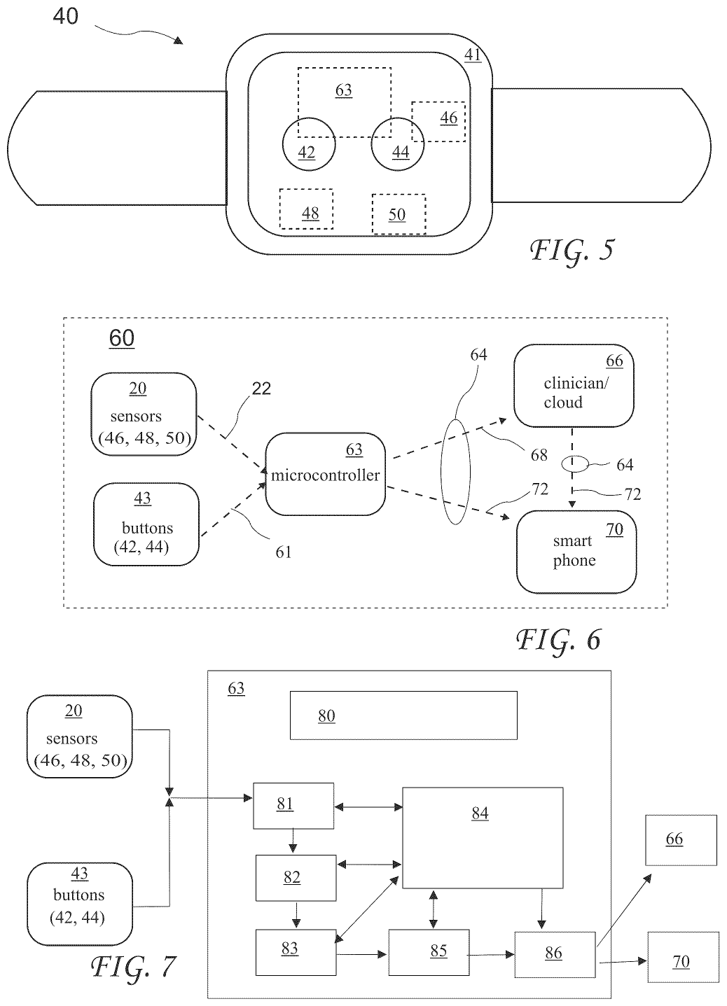

A wearable device 40 is shown in FIG. 5 and elements of a stress or cravings monitoring system 60 are shown in FIG. 6. The wearable device 40 includes housing 41 including sensors 20, for example, a 1, 2, or 3-axis accelerometer sensor 48, a thermopile sensor 50, and a pulse oximeter sensor 46. The wearable device 40 further includes buttons 43, for example a subject stress input (e.g., subject button) 42, a subject craving input (e.g., subject button) 44, and a microcontroller (e.g., microprocessor) 63. The sensors 20 provide sensor signals 22 to the microcontroller 63 and the buttons 43 provide button signals 61 to the microcontroller 63.

The wearable device 40 collects and processes physiological parameters and subject inputs to anticipate substance abuse by monitoring for the physiological parameters associated with stress leading to craving and detects substance abuse by monitoring for physiological parameters associated with substance abuse. The system 60 includes the wearable device 40 which may be coupled to a clinician and/or cloud 66 or operate autonomously, and coupled to the subject's smart phone 70 over a bluetooth connection or the Internet 64. The wearable device 40 may directly send alerts 72 to the subject though the bluetooth/internet 64 to the smart phone 70 to alert the subject to the onset of physiological conditions which may lead to harmful behavior. The wearable device 40 may also send messages 68 to the clinician/cloud 66 though the bluetooth/internet 64, to send alerts 72 to the subject though the bluetooth/internet 64 to the smart phone 68 to alert the subject to the onset of physiological conditions which may lead to harmful behavior. The wearable device 40 may further send messages 68 to the clinician/cloud 66 indicating a pattern of substance abuse and craving in the subject and for learning.

The thermopile sensor 50 detects temperature variability of the subject in given period of time. As a result, the temperature values are scheduled in such a way that the current temperature value is being compared with the previous acquired value constantly to check for the rate of change and corresponding variability. The 1, 2, or 3-axis accelerometer sensor 48 monitors motion of the subject on daily basis. This motion may show that the subject has reported stress and craving in spite of maintaining an active lifestyle. The pulse oximeter sensor 46 is a medical grade sensor which measures the heart rate of the subject on daily basis. The subject stress and craving input buttons 42 and 44 values indicate that the subject experience stress or cravings at a point of time. The subject inputs are recorded along with the timestamp at which the subject hits the input buttons 42 or 44.

Examples of features for detecting the stress or cravings and substance abuse are gross body movement measured by the 1, 2, or 3-axis accelerometer sensor 48, temperature variability measured by the thermopile sensor 50, and heart rate measured by the pulse oximeter sensor 46. The subject may also input stress and craving which may be correlated with the sensor measurements to monitor substance abuse of the subject. For example, if the subject records stress or craving, the corresponding sensor measurements are labeled as “substance event”, otherwise measurements are labeled as “normal event”. If a similar set of sensor measurements are detected, but there is no subject input at a particular time instance, then they are marked for “vulnerable event”. The clinician periodically reviews this set of sensor measurements to detect a pattern of substance abuse and craving in the subject. While a preferred sensor suite is shown in FIG. 5, data collection has shown that stress, cravings, and/or drug use may be identified with as few as one of the sensors, and a system with at least one of the sensors in FIG. 5 is intended to come within the scope of the present invention.

An example of the purpose of obtaining the subject inputs at a given time instance is to mark the number of times the subject is stressed which might lead to substance craving. Hence the following subject input protocol is maintained to effectively derive at the proposed hypotheses based on subject input values: a simple questionnaire to mark the stress and anxiety levels of the subject before using the wearable is recorded; a preliminary assessment on the substance abuse of the subject before using the wearable is recorded; the subject is informed of their accountability in pressing the input buttons whenever they feel stressed or possess substance craving at any time of the day; and the subject is informed of subject rewards such as points that are given to the subject every time a pattern of a healthy active lifestyle is detected, including regular exercise or providing reliable subject inputs on stress and cravings.

A microcontroller 63 of the wearable system 60 is shown in FIG. 7. The architecture design of microcontroller 63 provides formatting, labeling and aggregating the collected data at the wearable 40. The data collected during the “supervised period”, is triggered whenever the subject presses the buttons 43, for example, the button 42 or 44 to mark “stress” and “craving”. The training data collection may be for a fixed period of time after the button 42 or 44 is pressed, for example, preferably between 10 and 30 minutes or more preferably about 20 minutes, or most preferably 20 minutes. The training data collection may also be based on sensor measurements, where the sensor measurements indicate the presence of stress or cravings. The training data is used to train the algorithm embedded in the wearable. During the unsupervised period, the algorithm detects stress or cravings at the wearable and provides alerts to the subject directly through the smart phone 70. Using the microcontroller 63, the wearable device 40 may perform computing and control without requiring the Internet based (Internet of Things), not requiring computation at a remote server.

The microcontroller 63 includes a microprocessor 84, a battery management unit 80, a data accumulator 81, a variability processor 82, a data aggregating unit 83, a buffer 85, and an output module 86. The battery management unit 80, includes a regulator along with battery management system that controls wearable 40 providing a longer period of operation.

The microprocessor 84 is a general purpose processor that can perform basic arithmetic and logic operations. The microprocessor 84 has certain limitations in terms of the resources, when performing advance computations and data processing as these tasks involve storing, comparing, analyzing, aggregating, and processing the collected data. To achieve the processing requirements, the data accumulator 81, the variability processor 82, the data aggregating unit 83, and the buffer 85, communicate with the microprocessor 84 in our custom-built integrated chip. The purpose of the microprocessor 84, the data accumulator 81, the variability processor 82, the data aggregating unit 83, and the buffer 85, is to perform the data analysis tasks at the wearable device in a timely and efficient manner (termed as edge intelligent).

The data accumulator 81 collects data from the sensors 46, 48, and 50 and stores the acquired data as data packets with information that includes but not limited to Subject ID, Data, time, sensor data. The data accumulator 81 collects the data from the sensors 20 and stores the collected data in a specified data format. The input for the data accumulator 81 is raw sensor data and the output is the data packets.

The variability processor 82 receives data packets from the data accumulator 81 and computes variability in the data between time t and t+1. In each data packet, the changes in the sensor data is compared for the given subject ID, with respect to date and time. The variability processor 82 helps in calculating the variability in the features between time t and t+1. In each data packet, the changes in sensor information is compared for the given subject ID, with respect to date and time. The variability processor 82 includes adders, subtractors and multipliers which can help in performing the tasks.

The data aggregating unit 83 receives the variability in the data from the variability processor 82 and, calculates the state of the subject and tags events that can be used for unsupervised learning. The machine learning algorithm is applied to the data set in the data aggregating unit 83. The data aggregating unit 83, helps in calculating the state of the subject and tagging the events that can be stored in the databases for unsupervised learning. The machine learning algorithm is applied to the data set in the data aggregating unit 83. The data aggregating unit 83 is one example of a hardware realization of the implemented algorithm.

The first processing is to see whether the data set (i.e. sensor information) at time “t” has any kind of variability in comparison to “t+1”. After the initial data processing done through the data accumulator 81 and the variability processor 82, the data proceeds to the data aggregating unit 83 to apply the machine learning algorithm. These steps provide faster data processing needed for an edge-intelligent based wearable.

The microprocessor 84, receives data from the sub-blocks 81, 82, and 83 to perform arithmetic and logic operations and sends results to the buffer 85 to store and creates alerts transmitted through the output module 86. The buffer 85 stores the results and creates alerts transmitted by the output module 86.

The battery management unit 80, includes a regulator circuit that can provide fixed output voltage irrespective of any fluctuations and a battery management system that can monitor the battery performance and temperature over a period of time. The purpose of the battery management unit 80 is to ensure that the wearable is powered up for longer period of time.

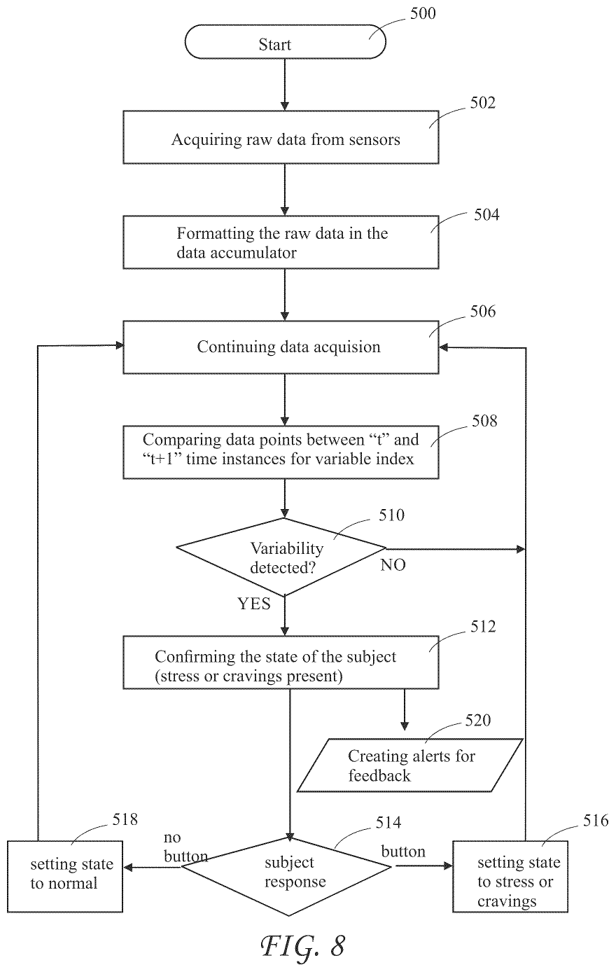

Processing performed in the wearable 40 is described in FIG. 8. The processing starts at step 500. Raw data is acquired from sensors at step 502. The raw data is formatted in the data accumulator 81 at step 504. Data acquisition continues at step 506. Data from time “t” is compared to data from time “t+1” for variable indices in the variability processor 82 at step 508.

If there are any changes in the features extracted through the sensors, the difference in these changes are tagged as “variability index”. In case of such changes in the features, 510 checks if there is a similar pattern from earlier data, and confirms the state of the subject in 512. If variability is detected at step 510 and the subject has pressed one of the buttons, the state is set to stress or cravings at step 516, if no button has been pressed, the state is set to Normal at step 518. Alerts are created for feedback at step 520.

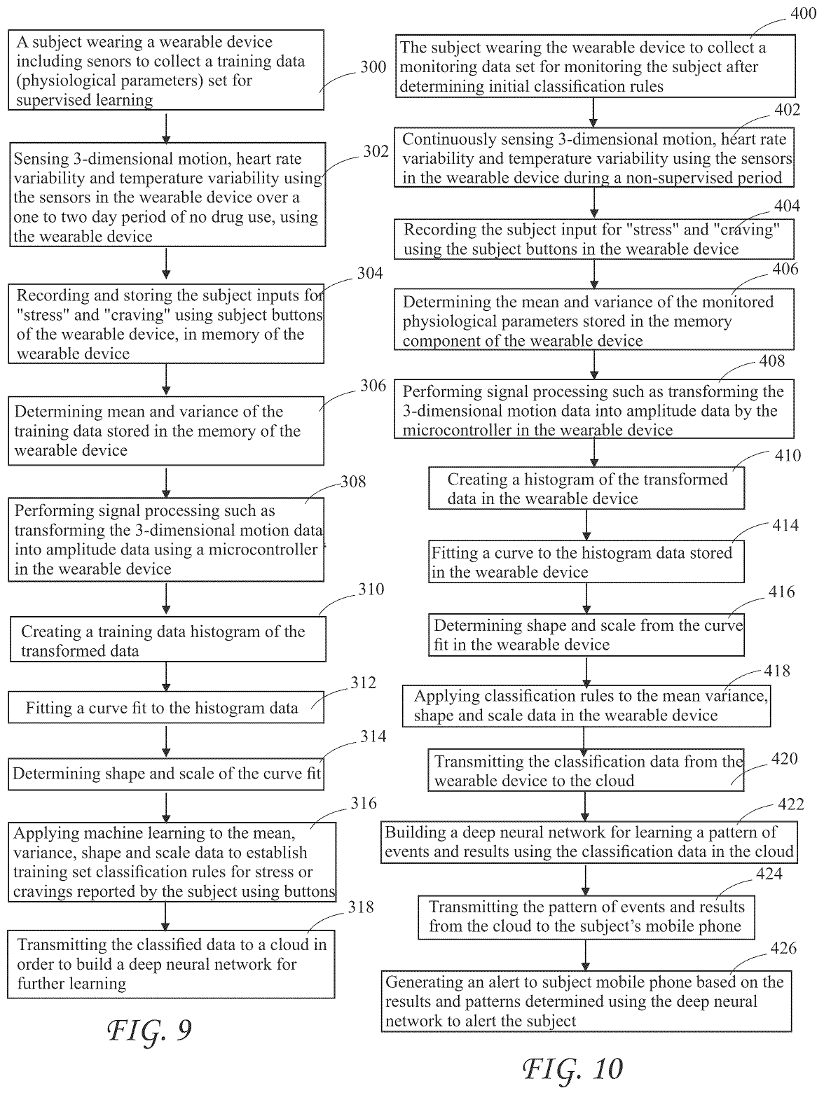

A method for collecting and processing a subject's physiological parameters during supervision is shown in FIG. 9. The method includes: a subject wearing a wearable device including senors to collect a training data (physiological parameters) set for supervised learning at step 300, sensing 3-dimensional motion, heart rate variability and temperature variability using the sensors in the wearable device over a one to two day period of no drug use, using the wearable device at step 302, recording and storing the subject inputs for “stress” and “craving” using subject buttons of the wearable device, in memory of the wearable device at step 304, determining mean and variance of the training data stored in the memory of the wearable device at step 306, performing signal processing such as transforming the 3-dimensional motion data into amplitude data using a microcontroller in the wearable device at step 308, creating a training data histogram of the transformed data at step 310, fitting a curve fit to the histogram data at step 312, determining shape and scale of the curve fit at step 314, applying machine learning to the mean, variance, shape and scale data to establish training set classification rules for stress or cravings reported by the subject using buttons at step 316, and transmitting the classified data to a cloud in order to build a deep neural network for further learning at step 318.

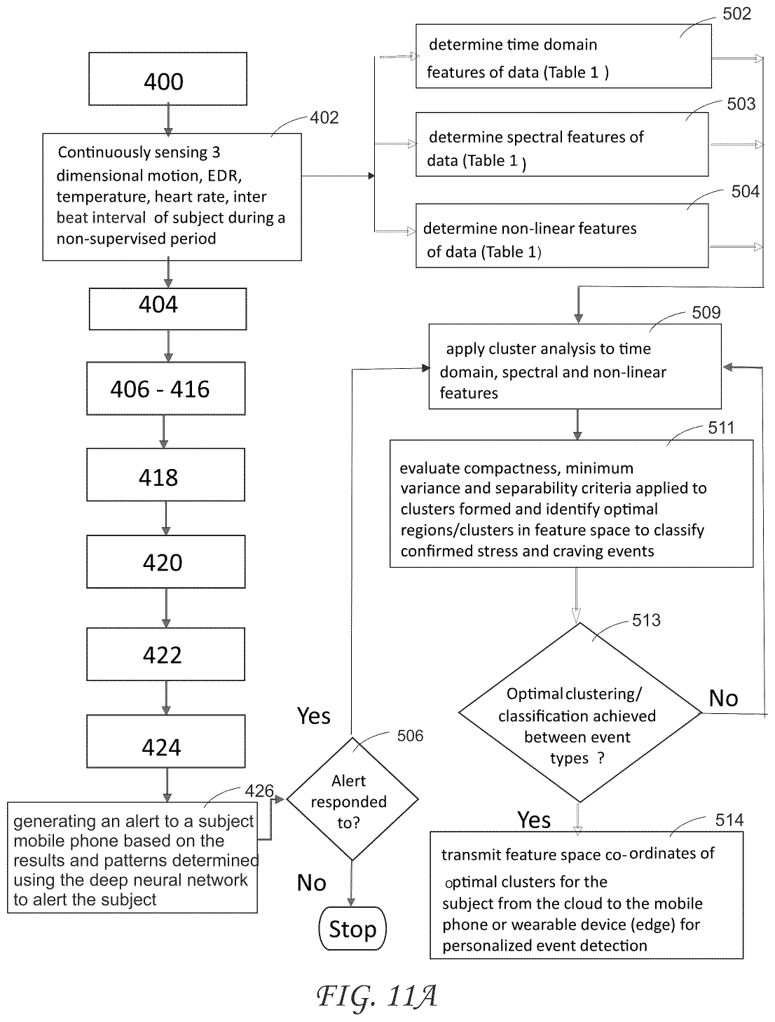

A first method for collecting and processing a subject's physiological parameters after supervised training and providing alerts, is shown in FIG. 10. The method includes: the subject wearing the wearable device to collect a monitoring data set for monitoring the subject after determining initial classification rules at step 400, continuously sensing 3-dimensional motion, heart rate variability and temperature variability using the sensors in the wearable device during a non-supervised period at step 402, recording the subject input for “stress” and “craving” using the subject buttons in the wearable device at step 404, determining the mean and variance of the monitored physiological parameters stored in the memory component of the wearable device at step 406, performing signal processing such as transforming the 3-dimensional motion data into amplitude data by the microcontroller in the wearable device at step 408, creating a histogram of the transformed data in the wearable device at step 410, fitting a curve to the histogram data stored in the wearable device at step 414, determining shape and scale from the curve fit in the wearable device at step 416, applying classification rules to the mean variance, shape and scale data in the wearable device at step 418, transmitting the classification data from the wearable device to the cloud at step 420, building a deep neural network for further learning using the classification data in the cloud at step 422, transmitting a pattern of events and results from the cloud to the subject's mobile phone at step 424, and generating an alert to a subject mobile phone based on the results and patterns determined using the deep neural network to alert the subject at step 426.

The deep neural network processing in step 422 may alternatively be performed in the wearable device using an edge intelligent approach. In instances where insufficient data is available for the deep neural network processing, the smart phone alerts may be solely based on comparing non-supervised measurements to the classification rules.

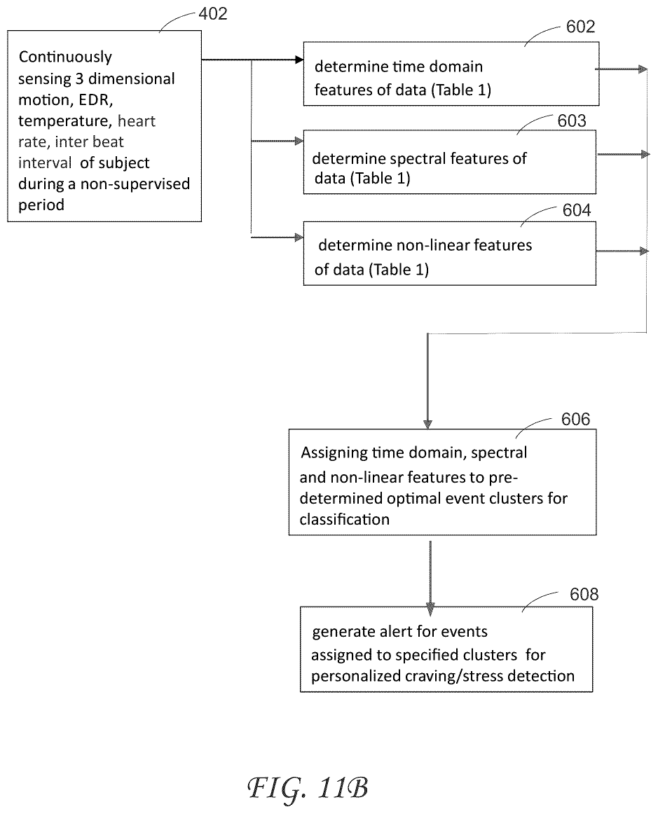

A first method for personalizing detection of cravings and stress in subjects using wearable sensor system is shown in FIG. 11A. The method includes: the subject wearing the wearable device to collect a monitoring data set for monitoring the subject after release from supervision at step 400 (see FIG. 10), continuously sensing 3-dimensional motion, EDR, temperature, heart rate, and inter beat interval of subject during a non-supervised period at step 402, determine time domain features of data per Table 1 at step 502, determine spectral features of data per Table 1 at step 503, and determine non-linear features of data per Table 1 at step 504. Proceeding from each of steps 502, 503, and 504 to apply cluster analysis to time domain, spectral and non-linear features at step 509.

TABLE 1
List of features for the accelerometry and heart rate signals.
Features for Accelerometry and Heart rate data

| Time based measures | Spectral measures | Nonlinear Measures |
|---|---|---|
| Mean | Mean Power in a predefined | Long - and short-range |
| | frequency band [f1, f2] | correlations in the sensor data |
| | f1 and f2 are the lower and | related to the signal variability, |
| | upper frequency limits used to | quantified by the Poincare plot |
| | estimate the power spectrum. | technique in which the sensor |
| Standard Deviation | Standard Deviation of power | data is plotted on a two- |
| | histogram | dimensional graph. |
| Skewness | Median Power | Derived measures: |
| Kurtosis | Total power in [f1, f2] | Major axis of overlaid ellipse (SD1) |
| Minimum | Peak power in [f1, f2] | Minor axis (SD2), SD1/SD2 |
| | | Area of the ellipse = SD1*SD2*pi |
| Maximum | Frequency at peak power | Signal correlation analysis - |
| | | variation with time lags, |
| | | characterization by detrended |
| | | fluctuation analysis, multiscale |
| | | entropy and such techniques |
| Energy | Spectral entropy | Pattern irregularity of the data |
| Root mean square | | determined using the entropy |
| Mean absolute error | | measures such as sample entropy, |
| | | approximate entropy and similar |
| | | methods. |

Feature for inter-beat interval data

| Time based measures | Spectral measures | Nonlinear measures |
|---|---|---|
| Standard deviation of | Frequency spectrum analysis | Characterizing underlying |
| successive differences | covering very low, low and high | structure by time delay |
| | frequency bands suitable for | embedding and estimating |
| | heart rate variability signal. | correlation dimension |
| Standard deviation of | | Pattern irregularity of the sensor |
| normal to normal (NN) | | data determined using the |
| intervals | | entropy measures such as sample |
| | | entropy, approximate entropy |
| | | and similar methods. |
| Root mean square of | Ratio of power between the | Long- and short-range |
| successive differences | low and high spectral regions | correlations in the inter beat |
| | | intervals quantified by Poincare |
| | | plot indices |
| Probability of intervals greater | | |
| than x msec and smaller than −x | | |
| msec | | |
| Usually, x = 50, 100 msec | | |
| Indices from the interval | | |
| histogram Triangular index | | |
| (TRI): reciprocal of the | | |
| probability of the | | |
| highest bin of the histogram of | | |
| RR intervals with a specified | | |
| bin size. | | |
| TINN is the width of the | | |
| triangular function, which best | | |
| fits the sample interval | | |
| histogram. | | |

Following step 424 (see FIG. 10), generating an alert to a subject mobile phone based on the results and patterns determined using the deep neural network to alert the subject at step 426, receiving a response to an alert from the subject at step 506, if NO, then stop, if YES, proceed to step 509. Then evaluate compactness, minimum variance and separability criteria applied to clusters formed and identify optimal regions/clusters in feature space to classify confirmed stress and craving events at step 511. Proceed to determine if optimal clustering/classification achieved between event types is analyzed at step 513, if NO, return to step 509, if YES, transmit feature space coordinates of optimal clusters for the subject from the cloud to the mobile phone or wearable device (edge) for personalized event detection at step 514.

An second method for personalized detection of cravings and stress in subjects using wearable sensor system is shown in FIG. 11B. The method includes: the subject wearing the wearable device to collect a monitoring data set for monitoring the subject after release from supervision at step 400 and continuously sensing 3-dimensional motion, EDR, temperature, heart rate, inter beat interval of subject during a non-supervised period at step 402 in FIG. 10, The method then determines time domain features of data per Table 1 at step 602, determine spectral features of data per Table 1 at step 603, and determine non-linear features of data per Table 1 at step 604. Directly following each of steps 602, 603, and 604, assigning time domain, spectral and non linear features to pre-determined optimal event clusters for classification at step 606, and generate an alert for events assigned to specified clusters for personalized craving/stress detection at step 608.

The computations in FIGS. 11A and 11B may be performed in a mobile phone or wearable (edge) device.

While the methods of the present invention specifically target cravings for drugs, similar methods may be developed and applied to other disorders. For example, eating disorders/food addiction, co-occurring disorders with addiction, and suicidal ideation associated with Post-Traumatic Stress Disorder (PTSD) and other such mental health conditions. Those skilled in the art will recognize that the methods of the present invention directed to these other disorders come within the scope of the present invention.

While the invention herein disclosed has been described by means of specific embodiments and applications thereof, numerous modifications and variations could be made thereto by those skilled in the art without departing from the scope of the invention set forth in the claims.

I claim:

1. A method for improving detection of cravings and stress in a subject after supervised training, the method comprising:
developing classification rules for detection of cravings and stress in the subject;
collecting subject physiological data;
applying the classification rules to the subject physiological data;
providing an alert to the subject when the classification rules applied to the subject physiological data indicates the stress or cravings are present;
the subject responding either agreeing or disagreeing with the alert;
when the subject agrees with the alert:
collecting features of the subject physiological data associated with the alert; and
forming clusters of the features of the subject physiological data; and
after completing the forming clusters, generating cluster based alerts when new subject physiological data falls into one of the clusters.

2. The method of claim 1, wherein collecting the subject physiological data comprises collecting time domain features, spectral features, and non-linear features.

3. The method of claim 1, wherein the forming the clusters comprises a k-means method.

4. The method of claim 1, wherein the forming the clusters further includes evaluating compactness, minimum variance and separability criteria applied to the clusters formed and identify optimal regions/clusters in feature space to classify confirmed stress and craving events.

5. The method of claim 1, wherein the completing the forming clusters comprises forming clusters of the features of the physiological data 30 or more times.

6. The method of claim 1, wherein the providing the alert to the subject comprises forming clusters of the features of the physiological data 50 or more times.

7. The method of claim 1, wherein the providing the alert to the subject comprises forming clusters of the features of the physiological data 70 or more times.

8. The method of claim 1, wherein the providing the alert to the subject comprises forming clusters of the features of the subject physiological data 90 or more times.

9. The method of claim 1, wherein the clusters comprise a pre-specified number of the clusters.

10. The method of claim 1, wherein the features corresponding to subject physiological data related to an event are assigned to the cluster with which the subject physiological data is most similar based on a ‘distance’ metric chosen appropriately for an applied clustering method.

11. The method of claim 1 wherein the response data is combined with this, to identify the clusters into which the subject's confirmed cravings are assigned to and these regions will be specific for the subject.

12. The method of claim 1, wherein the forming the clusters of the features of the subject physiological data is optimized based on the evaluation of separability criteria of the clusters.

13. The method of claim 1 wherein the forming clusters and generating the cluster based alerts provides a personalized machine learning model for stress or craving detection.

14. The method of claim 13 wherein the personalized machine learning model for stress or craving detection is hosted on the wearable.

15. The method of claim 13 wherein the personalized machine learning model for stress or craving detection is hosted on the cloud.

16. The method of claim 1, wherein developing the classification rules comprises:
performing personalized training at a treatment facility or in the real world to characterize a presence of stress or cravings in the subject;
the subject wearing the wearable device to collect a monitoring data set for monitoring the subject after release from supervision;
continuously sensing subject data comprising 3-dimensional motion, EDR, temperature, heart rate, inter beat interval of subject during a non-supervised period;
determine time domain features of the subject data;
determine spectral features of the subject data; and
determine non-linear features of the subject data.

17. A method for personalized detection of cravings and stress in subjects using the wearable sensor system, the method comprising:
performing personalized training at a treatment facility or in the real world to characterize a presence of cravings in the subject;
the subject wearing the wearable device to collect a monitoring data set for monitoring the subject after release from supervision;
continuously sensing subject data comprising: 3-dimensional motion, EDR, temperature, heart rate, inter beat interval of subject during a non-supervised period;
determine time domain features of the subject data determine spectral features of the subject data;
determine non-linear features of the subject data;
assigning time domain, spectral and non linear features to pre-determined optimal event clusters for classification; and
generate an alert for events assigned to specified clusters for personalized craving/stress detection.

18. A method for improving personalized detection of cravings and stress in subjects using a wearable sensor system, the method comprising:
generating an alert to a subject mobile phone based on the results and patterns determined using the deep neural network to alert the subject;
receive response from the subject;
apply a clustering technique for identifying feature space regions of personalized events to data features of time domain, spectral, and non-linear features;
evaluate compactness, minimum variance and separability criteria applied to clusters formed and identify optimal regions/clusters in feature space to classify confirmed stress and craving events;
optimal clustering/classification achieved between event types is analyzed; and
transmit feature space coordinates of optimal clusters for the subject from the cloud to the mobile phone or wearable device (edge) for personalized event detection.
